(19) United States
(12) Patent Application Publication
(10) Pub. No.: US 2022/0226583 A1
(43) Pub. Date: Jul. 21, 2022

(54) Apparatus for Detecting a Dose of Medicament Delivered from an Injection Device

(71) Applicant: Sanofi, Paris (FR)

(72) Inventors: Michael Helmer, Frankfurt am Main (DE); Maurice Toporek, Frankfurt am Main (DE)

(21) Appl. No.: 17/617,098

(22) PCT Filed: Jun. 12, 2020
(86) PCT No.: PCT/EP2020/066374

§ 371 Date: Dec. 7, 2021

(30) Foreign Application Priority Data
Jun. 13, 2019 (EP) 19305752.8

(51) Int. Cl. A61M 5/315; A61M 5/32

(52) U.S. Cl. CPC A61M 5/31568; A61M 5/31511; A61M 2005/3125; A61M 5/3202; A61M 5/31585

(57) ABSTRACT

In order to address a common need for cost efficient capturing of data relative to dosage and administration time in self-medication schemes, the present disclosure proposes an apparatus with an accessory and an injection device, wherein the accessory attaches and detaches to the injection device, the accessory housing a control unit; and the injection device includes a passive electronic arrangement and the control unit is configured to use the passive electronic arrangement to determine information correlating to a dose of medicament injected.

### CROSS REFERENCE TO RELATED APPLICATIONS

The present application is the national stage entry of International Patent Application No. PCT/EP2020/066374, filed on Jun. 12, 2020, and claims priority to Application No. EP 19305752.8, filed on Jun. 13, 2019, the disclosures of which are incorporated herein by reference.

### TECHNICAL FIELD

The present disclosure relates to an apparatus for detecting a dose of medicament delivered from an injection device and in particular to an apparatus including a disposable injection device.

### BACKGROUND

A variety of diseases exists that require regular treatment by injection of a medicament. Typically a medical practitioner formulates a dosage regime that manages the timing and dosage of the injections a patient should follow. Thus the timing and/or the dosage of the injections can vary between patients and between injections. Often, as part of the dosage management regime, users are required to record parameters of the injections, for example to monitor effectiveness of the treatment or as feedback during the calculation of parameters for subsequent injections. This could be achieved through the keeping of a manual data logbook.

The injections can be performed either by medical personnel or by patients themselves by using injection devices. Injection devices (i.e., devices capable of delivering medicaments from a medication container) typically have a syringe connected to a medicament container and a dose dispensing mechanism for driving the medicament through the needle. The medicament chamber may be re-useable wherein the dose dispensing mechanism is designed to be reset, allowing an empty medicament cartridge to be replaced by a new one. Alternatively, the injection device may be disposable wherein, upon the contents of a pre-filled medicament container being emptied, the injection device is disposed of. Suitably, the injection device includes a dose setting mechanism that allows a user to set or ‘dial in’ an amount of medicament to be administered.

As an example, type-1 and type-2 diabetes can be treated by patients themselves by injection of insulin doses according to a dosage regime, for example injections once or several times per day. WO2004/078241 discloses a suitable injection device, typically referred to as a pen, and references to pen herein are interchangeable with injection device. It is known for a disposable pen to be provided with a set of one-way needles that are attached to the pen before each use. The insulin dose to be injected and prescribed by the dosage regime can then, for instance, be manually selected through the dose setting mechanism by turning a dose knob to the required volume. The dose is then injected by inserting the needle into a suited skin portion and pressing an injection button of the dose dispensing mechanism. As part of the management of the dosage regime, the user records parameters of the injection. Such parameters, for instance, may be one or more of; the date and time of injection, blood sugar results, medication and dose, and/or diet and exercise information.

### SUMMARY

According to general aspects of the present specification, there is provided an apparatus including an injection device and an accessory for the injection device. The injection device includes a passive electronic arrangement that can be interrogated to determine information relating to a position of a part of the injection device pre-injection and post-injection as part of a dose tracking mechanism. The accessory includes a control unit to provide a signal to the passive electronic arrangement. The accessory is removable from the injection device. The apparatus therefore takes advantage of the reuse of resources by reusing the control unit between injection devices. Thus, the costs and resources of the control unit are shared between multiple injection devices. The additional costs associated with providing the injection device with the passive electronic arrangement can be relatively low.

In the exemplary embodiments, the passive electronic arrangement on the injection device does not generate energy; rather, the control unit provides the necessary energy to the passive electronic arrangement. An electrical parameter of the passive electronic arrangement is based on the position of said part of the injection device. When the passive electronic arrangement is provided with energy by the control unit, the control unit uses the electrical parameter of the passive electronic arrangement to correlate to a dose of medicament injected. With the injection device only including a passive electronic arrangement, the passive electronic arrangement is not powered without interaction with the accessory. For instance, the control unit of the accessory powers the passive electronic arrangement by applying a signal, for instance a voltage or a current, to the passive electronic arrangement. Or, for instance, by applying a signal through inductive coupling between the control unit of the accessory and the passive electronic arrangement.

In exemplary embodiments, the accessory may electrically connect to the injection device when the accessory is installed on the injection device. Here, the control unit is electrically connected to the passive electronic arrangement through a connector. Alternatively, in some embodiments, the control unit is inductively coupled to the passive electronic arrangement.

In exemplary embodiments, the accessory replaces a cap of said injection device. Here, the accessory is a cap and covers a part of or a substantial part of a medicament reservoir such as a cartridge or a cavity for holding a cartridge. By using the accessory as a cap to cover and protect a distal end of the injection device, removing and replacing the accessory before and after an injection can be used as a trigger to cause the control unit to interrogate the passive electronic arrangement. For instance, the accessory includes a switch that is activated between states by relative movement of the accessory and injection device. Here, one of the accessory and injection device may include a catch and the other a cooperating part such as a projection. The catch catches and releases on the projection during the attachment and detachment of the accessory from the injection device. The switch is therefore pushed and pulled between states by the relative movement between the accessory and the injection device. Therefore, in exemplary embodiments, the accessory includes a switch that is toggled between on and off states by attachment and detachment of the accessory from the injection device.

The accessory is suitably attached and detached from the injection device by relative linear movement in an axial direction. For instance, the accessory may attach and detach to the injection device substantially in accordance with the attachment and detachment of known caps. For instance, the attachment and detachment may additionally include a threaded connection between the parts. Here, the attachment and detachment is achieved, at least in part, by a relative rotation of the parts to engage threads of the threaded connection. In addition to optionally activating a switch, said relative movement connects and disconnects the connector. Here the connector on the accessory is connected and disconnected from a corresponding connector on the injection device. In embodiments with linear movement, the connector on the accessory may slide into contact with the connector on the injection device. The contact may be through abutment in the direction of relative movement of the accessory and injection device. Alternatively the abutment may be in a direction transverse the relative movement of the accessory and injection device and a resilient nature of one or both of the connectors or one or both of the parts the connectors are mounted on causes a pressing abutment between the connectors.

Suitable connectors may be applied. In one exemplary embodiment the connectors are electrode pads. The electrode pads in the accessory are connected to the control unit. The electrodes in the injection device are connected to the passive electronic arrangement. The connections may be wires or conductive tracks or any other suitable conductive line.

The accessory may include an optional display. The control unit controls the display to display information regarding the injection dose regime and may include the dose tracking information. The control unit may process the pre-injection and post-injection information to determine the dose as part of the dose tracking mechanism. Alternatively, the control unit may include a communications module and the communications module may transmit the information to a remote device for processing. Additionally or alternatively, the remote device may run an application or program to monitor and alert a user to an injection regime. Here, the communications module may communicate with the remote device and may display information relating to future injections, for example dose and time. By housing the display and/or the communications module on the accessory, the display and communications module can be reused between injection devices.

The accessory suitably includes a body that houses the connector and control unit. Suitably, the body forms a cap to cover a part or a portion of the injection device. Here, the body includes a closed recess that accepts a distal end of the injection device. Suitably, the closed recess covers a substantial portion of the injection device's cartridge. The body may also house the optional display. The body may include a compartment for housing the power supply, for instance a battery compartment and battery. By housing the power supply on the accessory, the passive electronic arrangement on the injection device is provided with an electrical signal through the connector. Thus the passive electronic arrangement is not provided with an electrical signal when the accessory is unattached.

According to the exemplary embodiments and a further aspect, there is therefore provided an apparatus including the accessory and an injection device. The accessory includes an optional connector and a control unit. The injection device includes a passive electronic arrangement and in some embodiments a corresponding connector. In some embodiments, the control unit is arranged to provide a signal to the passive electronic arrangement via electrical connection between the respective connectors. For instance the signal is a voltage or current. Here, attaching the accessory to the injection device simultaneously connects the connector of the accessory to the connector of the injection device. Alternatively, the control unit is inductively coupled to the passive electronic arrangement. Here, attaching the accessory to the injection device brings the respective inductive components into proximity.

In the exemplary embodiments, the control unit includes an active electronic arrangement. Suitably, the active electronic arrangement includes a battery or other power or energy source. The connection of the active electronic arrangement via the connectors or via the inductive coupling to the passive electronic arrangement forms an electrical circuit. Here, the control unit measures an electrical parameter of the circuit. As described herein, the passive electronic arrangement is such that an electrical parameter of the circuit changes as a position of a part of the injection device changes, and the value of the electrical parameter is representative of the position of the part of the injection device. For instance, the electrical parameter may be a resistance or a capacitance or an inductance of the circuit and specifically of the passive electronic arrangement.

In one exemplary embodiment, the passive electronic arrangement includes a variable electronic resistor. Here, a conductive track provides a resistive path between two terminals. One or more wipers are arranged to move along the conductive track in response to movement of a part of the injection device that moves during an injection process. The wiper or wipers connect to one or more of the terminals such that the length of the conductive track between the terminals changes thereby increasing or decreasing the resistance of the passive electronic arrangement, wherein the change in resistance can be correlated to a movement or position of the part. Here, the control unit applies a signal having a voltage to the passive electronic arrangement and measure the current to determine the resistance or change in resistance of the passive electronic arrangement. The variable electronic resistor can be interrogated by the control unit applying a signal to the passive electronic arrangement pre-injection and post-injection and the difference in resistance used to correlate to a displacement of the part. The displacement of the part between a pre-injection position and a post-injection position can further correlate to a dose dispensed. The dose tracking mechanism includes calculating a dose based on the displacement of the respective part.

In an alternative exemplary embodiment, the passive electronic arrangement includes a capacitive sensor. Here, the control unit applies a signal across two plates of the capacitive sensor that are separated by a gap. The plates of the capacitive sensor are provided by metallic, electrically conductive elements. The plates may be printed, or they may be cut from a sheet, or formed in any other suitable way. As will be apparent from the following description, the plates may not be planar but instead may be curved. By arranging a part of the injection device being monitored in the gap between the plates, the capacitance of the passive electronic arrangement can be caused to change based on a movement or position of the part. The control unit measures changes in the capacitance of the passive electronic arrangement (by applying a signal to the passive electronic arrangement and detecting how it responds) and correlates the measurement to a movement or position of the part.

Suitably, the two plates of the capacitive sensor are arranged to either side of a part of the injection device. Said part includes a volume, the contents of which change in response to movement of a part during the injection process. For instance, the passive electronic arrangement may be arranged about a cartridge containing liquid medicament. The dose tracking mechanism includes calculating a dose by correlating a change in capacitance between the pre-injection and post-injection measurements that results at least in part from the change in the quantity of liquid medicament present in the volume between the plates of the capacitive sensor.

Alternatively, the dose tracking mechanism includes calculating a dose by correlating a change in capacitance between the pre-injection and post-injection measurements that results from a different location of plastic and/or metal parts in the volume between the plates.

Suitably, the plates extend along a longitudinal axis of the injection device. In some embodiments, the plates are formed within a label that is applied to the injection device. For instance, the plates may be integrated into the information label that is applied to the injection device and carries user-readable information regarding the medicament. Here, electrodes or terminals can also be optionally formed on the label for electrically connecting the control unit to the passive electrical arrangement. Advantageously, by forming the passive electronic arrangement as a capacitive sensor having plates formed in a label, the label can be applied to retrofit to an existing injection device. Thus, other than the label, the other parts of the existing injection device are wholly unchanged or substantially unchanged.

In some embodiments, an RFID device, which typically includes an RFID chip and an antenna formed by an electric circuit is included. In operation, when the RFID device is in the reach of a reader device with an RFID reader, the antenna receives a signal from the reader device and sends a wireless response signal according to the information encoded in a memory of the RFID chip.

In a representative example including an RFID device, the electric circuit of the antenna is in a closed circuit (e.g., completing the circuit and enabling the antenna to transmit the response signal) with the passive electronic arrangement. As explained herein, the passive electronic arrangement is configured to be operatively coupled with the movement of one or more components of the injection device. In this manner, when a position of said component of the drug delivery device (e.g., part of a dose dispensing mechanism) changes during, a corresponding change in the configuration of the passive electronic arrangement in an RFID device's electric circuit is made. As a result, the resonance frequency of the RFID device changes and this change indicates the change in position of the component. The change in position is therefore an indication of a dose dispensed during the dose dispensing operation.

For example, if 10 units of a medicament is delivered from an injection device with a corresponding movement of a dose dispensing mechanism, the passive electronic arrangement is adjusted by an amount corresponding to the 10 units, and this, in turn, causes a change in the resonance frequency of the RFID device that indicates 10 units of change of the dose dispensing mechanism. As an illustrative example, an RFID device has a default resonance frequency of 13.00 MHz and a passive electronic arrangement in the circuit of the RFID device is coupled to a dose dispensing device such that change in position of the dose dispensing mechanism changes the resonance frequency by +0.1 MHz for every unit of dose dispensed by the dose dispensing device by changing a property of the electric circuit of the RFID device (e.g., resistance, capacitance, or inductance). Therefore, after dispensing 10 units of dose (and before the dose dispensing device's position is reset), the resonance frequency of the RFID device is changed to 14.00 MHz. This new resonance frequency, as read by an RFID reader, is usable as an indication that 10 units of dose were dispensed from the drug delivery device.

In some embodiments the RFID chip is formed in part on the accessory and connected to the passive electronic arrangement through electrical connectors. Alternatively, the RFID device's circuit may be formed on the injection device, for instance within the label, and the control unit may include an RFID reader to inductively couple to the RFID device.

According to the exemplary embodiments and a further aspect, there is therefore provided an apparatus including the accessory, an injection device, and a processor for signal processing. The processor may be integral to the accessory, for instance, housed in the body, or the processor may be remote from the accessory, for instance in the remote device. In exemplary embodiments having a processor in a remote device, the accessory includes a communication module for communicating the information to the remote device such as a smartphone, tablet computer, smartwatch or other independent device (e.g., laptop or PC). In some exemplary embodiments, the control unit controls the communication module to transmit the acquired information to the remote device for processing. Thus the main electronic processing is completed by the remote device, utilising the electronics of the remote device that does not then need to be replicated on the accessory. Suitably, a power supply powers the control unit and enables the controller to provide a signal to the passive electronic arrangement via the connectors and communications module and display as required. Suitably, the power supply is housed in the body of the accessory.

In the exemplary embodiments, a processor completes processing steps to calculate a displacement of the respective part that moves during the injection process. The processer may convert the displacement into a dispensed dose measurement dependant on the part being monitored. Where the processor is remote from the accessory, the processor may cause the measurement to be transmitted to the accessory. Here, the control unit includes a communication module for receiving the dose measurement. A controller controls the communication module and the controller may control the communication module to communicate the delivered dose to a display. Here, the display is housed in the body of the accessory. The display is arranged to face the user and may be opposed to the connector. A power supply is provided to power the display, controller and communication module. The display may also display other information received by the communications module, for instance the time or dose of a next injection. Additionally or alternatively, the processor may transmit the dose measurement for storage in an electronic log book. For instance, the processor may communicate with an electronic log-book program or the like or the processor may process the images as part of an integrated electronic log-book program.

In some exemplary embodiments, the accessory includes one or more switches. The switches may interact with a controller of the control unit to initiate when to interrogate the passive electronic arrangement and when to transmit information via a communication module to a remote device, or to a display. The switches may be manually operated to indicate events before and after an injection has occurred. Alternatively, one or more switches may be automatically activated to indicate one or more of the events in the injection process. For instance, a switch may be automatically activated to indicate the removal of the accessory from the injection device or the operation of the dose dispensing mechanism or dose setting mechanism.

In the exemplary embodiments including a controller, the controller may include a memory to store information from one or more interrogations of the passive electronic arrangement as well as ancillary information such as the time and date of the interrogation.

In the exemplary embodiments including a communication module, the communications module may be a wireless communications module. For example, the wireless communication module is a short distance communication module. The method of operation may include the user completing a pairing step to pair the communication module to the remote device to establish a unidirectional or bidirectional mode of communication.

An axial direction is a direction along an axis of the injection device, for instance, coaxial with an axis of a syringe or the direction of movement of the medicament chamber's bung. In the exemplary embodiments, the accessory is attached to the injection device by attachment through relative movement between the accessory and respective injection device in the axial direction, and herein termed the engagement movement. The engagement movement may be in the axial direction in a proximal-distal direction or the reverse distal-proximal direction. Here, the proximal-distal direction is from the dose dispensing mechanism towards the cartridge and the distal-proximal direction is the opposite, from the cartridge towards the dose dispensing mechanism.

In exemplary embodiments, the accessory includes an attachment portion that restricts relative movement between the accessory and the injection device in a direction opposed to the engagement movement. In the exemplary embodiments, abutment between the attachment portion and injection device provides the restriction to the movement. Thus the attachment portion includes a means to attach the accessory to the injection device. Here, the attachment portion restricts relative movement between the accessory and injection device in at least one direction. Advantageously, the restricted movement allows the accessory to be physically attached and held to the injection device. Suitably the body includes the attachment portion.

In embodiments including a linear engagement movement in one of the axial directions, the attachment portion and injection device may be arranged to engage at a tapered portion. Here, at least one of the respective parts is tapered so that the parts are cooperatively engaged by friction. In one embodiment, the attachment portion is tapered. Here, the tapered portion of the attachment portion includes a first area forming opposed points spaced around the pen, and a second area having opposed points spaced around the pen, wherein the distance between the points of the first area is smaller than the distance between the points of the second area. Thus the restricted movement is provided by friction generated by a force applied by the engagement movement. Additionally or alternatively, the injection device is tapered at a connection area adapted to receive the accessory.

In additional or alternative embodiments including a linear engagement movement, the attachment portion and injection device may be arranged to provide a positive location. For instance, one of the injection device or the attachment portion includes a resilient portion over which the other part is arranged to pass. Here the resilient portion provides a localised restriction to a separation distance between the parts. Pushing the two parts together by the engagement movement and so that the respective part moves over the resilient portion produces a positive location that provides feedback to the user that the attachment of the accessory has been completed. It also provides initial resistance to the removal of the accessory in the reverse, disengagement movement direction.

In some embodiments, the attachment portion may include a locator to locate the accessory on the injection device. Suitably, the locator provides a rotation key to align the accessory and injection pen in a rotational alignment relative to the axial direction. Suitably, the key prevents rotational movement of the accessory relative to the injection device when attached via a linear engagement movement along the axial direction. Here, the attachment portion and injection device are arranged to have cooperating alignment features. The cooperating alignment features may include a non-symmetrical cross section relative to the axial direction or may include a protrusion and recess on respective parts. The locator aids the alignment of the respective connectors.

In the exemplary embodiments, the attachment portion engages with the injection device at a mid-point of the injection device. In exemplary embodiments, the accessory is adapted to attach to an injection device being a pen type device. The pen includes a medicament chamber, a dose dispensing mechanism, and a dose setting mechanism. Suitably, the dose dispensing mechanism and dose setting mechanism are assembled in a housing. Here, the housing may provide a connection to attach the medicament chamber, or the medicament chamber may also be assembled within the housing. In some exemplary embodiments, the housing is covered by an information label. Suitably, the information label may include a removable section to expose the window. For instance, the label may be formed with an area defined by perforations, and the user may remove the area defined by the perforations prior to attaching the accessory.

In the exemplary embodiments, the injection device includes a housing and the housing provides a receiving portion to receive an attachment portion of the accessory. The receiving portion cooperates with the attachment portion to locate and secure the accessory to the injection device. The receiving portion may include a locator to correspond to a locator of the accessory to aid alignment of the accessory to the window. Suitably, the receiving portion may be provided adjacent the medicament chamber or connection between the housing and medicament chamber.

According to an exemplary aspect, there is therefore provided an apparatus including an accessory and an injection device. The accessory includes a body that houses a connector and a control unit. The body defines an attachment portion for attaching the accessory to the injection device. The injection device includes a cartridge assembly assembled to a housing. The housing contains a dose dispensing mechanism and a dose setting mechanism. The dose dispensing mechanism includes a part that moves relative to a reference in response to movement of the dose setting mechanism. Wherein the attachment portion is specifically adapted to align the connector of the accessory to the connector of the injection device. The accessory and injection device may be provided as a kit of parts or individually for use with each other.

According to a further aspect, there is provided a method of managing a dosage regime. The method includes using the control unit of the accessory to provide a signal to the passive electronic arrangement before an injection and after an injection in order to determine a displacement of a part of an injection device and electronically logging the result as part of a does tracking mechanism. The method further includes attaching an accessory of the previous aspects to the injection device to form an apparatus of previous aspects.

The method may include operatively connecting an attachment portion of the accessory to a receiving portion of the injection device. The step of operatively connecting the attachment portion and receiving portion includes moving the attachment portion relatively to the injection device. The step of operatively connecting the accessory aligns a connector of the accessory to a connector of the injection device. The step of operatively connecting the attachment may include aligning and engaging a key on the accessory to a key on the injection device in order to rotationally align the respective connectors.

Suitably, the method includes steps of removing and replacing the accessory on to the injection device before and after an injection. Here, suitably, a switch is automatically activated by the removal and attachment of the accessory to the injection device. Suitably activation of the switch causes the control unit to provide a signal to the passive electronic arrangement.

The method may include a pairing step to pair the accessory with a remote device. For instance, to pair a communications module in the accessory with a corresponding module in the remote device. The pairing may be activated by operating a switch of the accessory or the like.

In the exemplary method, a pre-injection measurement step includes causing the control unit to provide a signal to the passive electronic arrangement, for instance by applying power to the passive electronic arrangement in order to measure a characteristic of an electrical parameter of the passive electronic arrangement. And a post-injection measurement step includes causing the control unit to provide a signal to the passive electronic arrangement, for instance by applying power to the passive electronic arrangement in order to measure a characteristic of an electrical parameter of the passive electronic arrangement. The pre-injection and post-injection measurement steps may be activated by operating a switch of the accessory. The switches may be operated manually or automatically upon another event. The measurement steps may include causing a communications module to transmit the information to a remote device. The information may be transmitted after completion of the injection or at stages throughout the injection.

In the exemplary embodiments, the method includes processing the information to calculate a displacement of the part of the injection device being monitored. The processing may be completed by a processor of a remote device. The processing suitably includes the step of converting the calculated displacement into a dose measurement.

The processor for the processing step may be part of a remote device. Here, the exemplary embodiments include a step of transmitting the pre and post measurement information to the processor. Suitably the transmission is completed by a communications module of the accessory. Here, the communications module may also be paired to the remote device to receive transmissions. For instance, the accessory may include a display wherein the method includes controlling the accessory to operate the display to display information received from the remote device.

In exemplary embodiments, the method includes a step of logging the dose calculation as part of an electronic log-book to record the dose measurement. The electronic log book may also log details of the injection such as the time of the injection based on the time of receipt by the processor of the information.

According to the exemplary embodiments, the method includes repeating the steps of logging a dose measurement of subsequent injections. The method further includes removing the accessory from a first injection device and reattaching the accessory to a second injection device. For instance, when a first injection device is emptied, the accessory is removed and replaced on a second, replacement injection device with medicament remaining therein.

According to the exemplary embodiments, there is therefore provided an improved accessory for an injection device, apparatus including the accessory and one or more injection devices, and a method of managing a dosage regime as set forth in the appended claims. Other features of the principal improvements will become apparent from the description and elsewhere in the application. By using an accessory to monitor the movement of a part of the injection device, a dose measurement can be electronically recorded as part of a dose management method. Moreover, by housing the control unit in the accessory, resources such as the communications module and power source can be shared between multiple injection devices.

### BRIEF DESCRIPTION OF THE FIGURES

Exemplary embodiments are described with reference to the accompanying drawings, in which:

FIG. 1 shows a schematic view of an injection device;

FIG. 2 shows an exploded parts view of the injection device of FIG. 1;

FIG. 3 shows a partial cross-sectional view through a grip end of the assembled injection device shown in FIG. 2;

FIGS. 4 and 5 are illustrations of parts of FIG. 3 showing a conductive track forming a variable electronic device for dose tracking;

FIG. 6 is a schematic illustration of an injection device and sensor components of the capacitive sensing of the status of an injection;

FIGS. 7 and 8 show perspective views of an accessory for an injection device from a front and rear view, respectively;

FIG. 9 shows a perspective view of an apparatus showing an accessory assembled to an injection device with an without an area shown removed for illustrative purposes;

FIG. 10 shows a schematic representation of a switch device for use with an apparatus including an injection device and accessory;

FIG. 11 details a schematic plan of a control layout; and

FIG. 12 shows a method of managing a dosage regime.

Reference will now be made in detail to the embodiments of the present disclosure, examples of which are illustrated in the accompanying drawings, wherein like reference numerals refer to like elements throughout.

### DETAILED DESCRIPTION

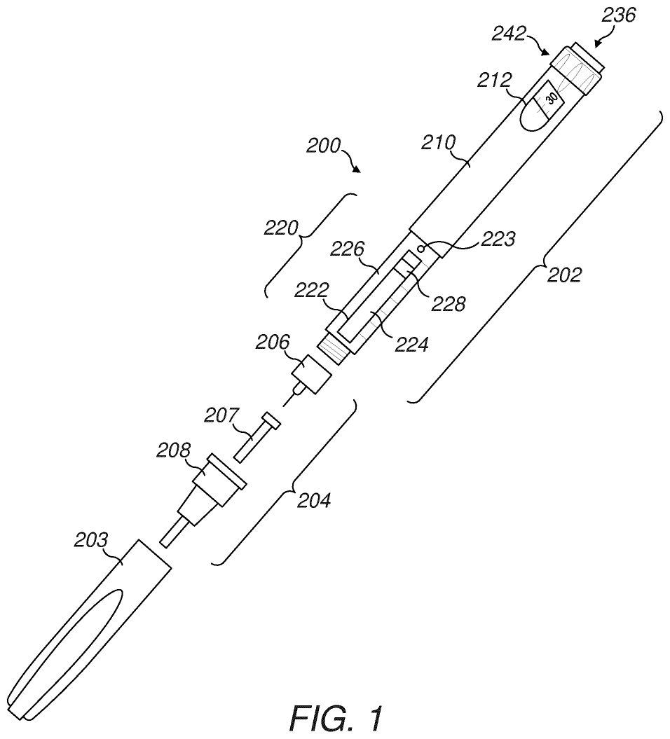

FIG. 1 is an exploded view of an injection device 200 suitable for use with exemplary embodiments. The injection device shown is often referred to as an injection pen or pen. Various designs of pen are known and whilst a brief description is given herein, it will be appreciated that the specific construction of the pen may alter and vary from the following description.

The injection device 200 has a distal end and a proximal end. The term “distal” refers to a location that is relatively closer to a site of injection, and the term “proximal” refers to a location that is relatively further away from the injection site.

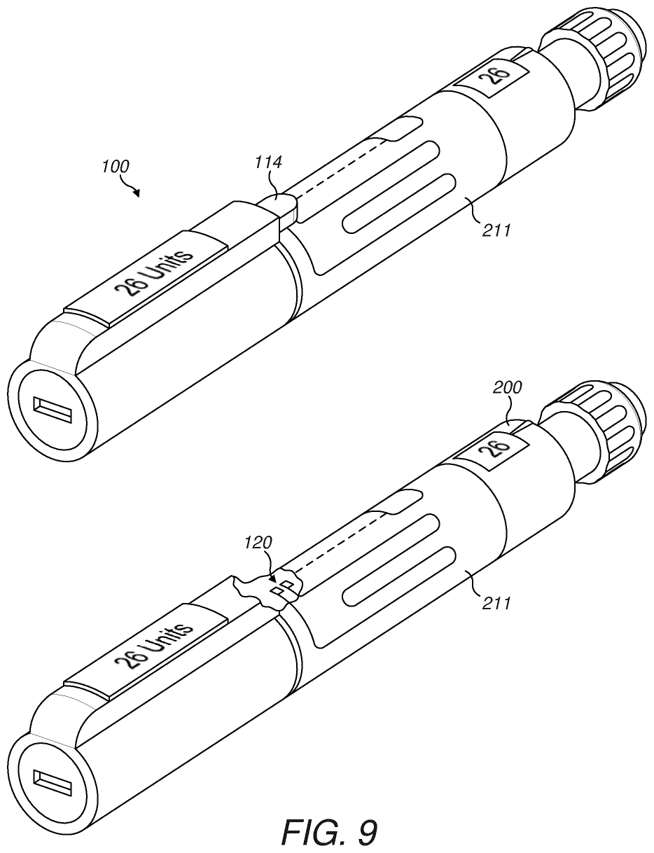

The injection device 200 includes a grip assembly 202, a cap 203 and a needle assembly 204. The grip assembly is formed from a housing 210 and a cartridge assembly 220. The cartridge assembly 220 includes a cartridge holder 222 for containing a cartridge 224 containing medicament. As shown, housing 210 is substantially cylindrical and has a substantially constant diameter along its longitudinal axis from a proximal end to a distal end. The longitudinal axis has a proximal-distal direction that extends from the proximal end to the distal end and the reverse distal-proximal direction. A label 211 (shown in FIG. 9) is provided on the housing 210. The label 211 includes information about the medicament included within the injection device 200, including information identifying the medicament. The information identifying the medicament may be in the form of text. The information identifying the medicament may also be in the form of a colour. The information identifying the medicament may also be encoded into a barcode, QR code or the like. The information identifying the medicament may also be in the form of a black and white pattern, a colour pattern or shading.

The cartridge assembly 220 is assembled to the housing 210 to form the grip assembly 202. Suitably, the proximal end of the cartridge assembly 220 includes a connection part (not shown) and the distal end of the housing 210 includes a corresponding connection part (not shown) that cooperatively engage with each other to connect the two parts. As shown, the cartridge holder 222 is substantially cylindrical with a hollow receiving for the cartridge 224. The cartridge includes a stopper 228 that can be advanced within the cartridge 224 during use to expel medicament from the cartridge 224. Here, it will be appreciated that the needle assembly 204 cooperates with the grip assembly to serve as a conduit for the medicament during injection.

The cartridge holder 222 has a porthole 226 in a side thereof. The porthole 226 allows the user to view the cartridge 224 through the porthole 226 when the cartridge 224 is contained in the cartridge holder 222. FIG. 1 shows a stopper 228 of the cartridge 224 visible through the porthole 226. FIG. 1 shows the cartridge holder 222 having one porthole 226, however, the cartridge holder 222 may instead have more than one porthole 226. For example, the cartridge holder 222 may have a first porthole 226 located on one side of the cartridge holder 222 and a second porthole located on a second, in some cases opposing, side of the cartridge holder 222. Thus a first side of the cartridge 224 within the cartridge holder 222 may be visible through the first porthole 226 while a second, different side of the cartridge 224 may be visible through the second porthole. Other porthole configurations may be used.

The needle assembly is shown including a needle 206, an inner needle cap 207 and an outer needle cap 208. A needle 206 of the needle assembly 204 can be affixed to the cartridge holder 222 such that the needle 206 is in fluid communication with the medicament in the cartridge 224. The needle 206 is protected by the inner needle cap 207 and the outer needle cap 208.

The removable cap 203 attaches to the cartridge assembly. The cap 203 at least partially covers the cartridge holder 222, and hence cartridge 224, when attached to the grip assembly. The cap 203 may also be attached to the grip assembly such that it at least partially covers the cartridge holder 222 with or without one or more of the needle 206, inner needle cap 207 or outer needle cap 208 being present.

The cartridge holder 222 may have a cap retaining feature 223 on an outer surface, for example adjacent a proximal end of the cartridge holder 222, and adjacent the attachment to the housing 210. Thus, the cap 203 may substantially cover the cartridge assembly when fitted. The cap retaining feature 223 engages with a corresponding coupling feature on an inner surface of the cap 203 to hold the cap 203 in place when attached to the grip assembly. The cap retaining feature 223 may include one or more of a ridge, groove, bump, lock and/or pip. In some examples, the cap retaining feature is located on the housing 210 of the injection device 200.

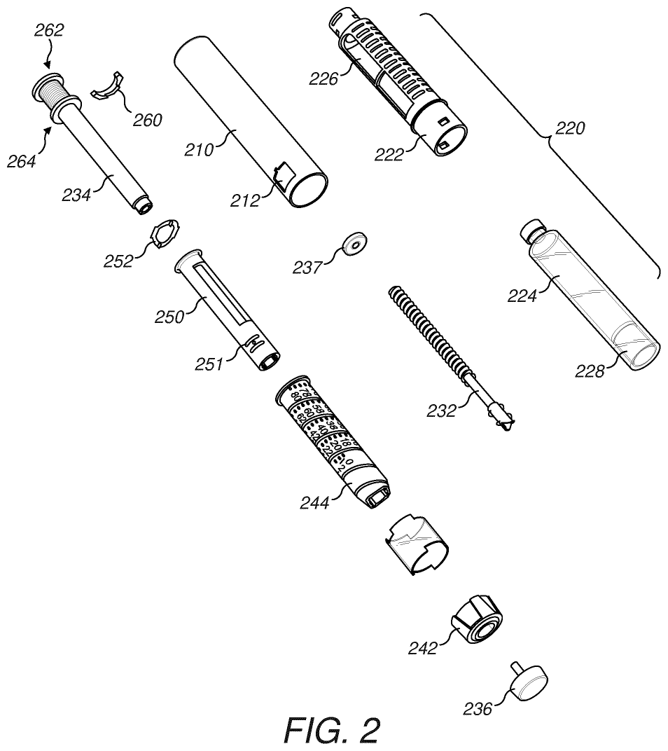

As shown in FIG. 2, the housing 210 houses a dose dispensing mechanism and a dose selection mechanism. The dose setting mechanism is used to select a dose to be injected and the dose dispensing mechanism is activated to inject the dose. In this instance, the dose dispensing mechanism is activated to drive the stopper 228 towards the distal end of the cartridge 224. The injection device 200 may be used for several injection processes until either the cartridge is empty or the expiration date of the injection device 200 (e.g. 28 days after the first use) is reached. Injection device 200 may be single-use or reusable.

To drive the stopper 228 into the cartridge 224, the dose dispensing mechanism includes a piston rod 232, a drive sleeve 234, and a trigger button 236, which act together to drive a pressure plate 237 against the stopper 228 and into the cartridge 224. A medicament or drug dose to be ejected from the drug delivery device 200 is selected by turning a dosage knob 242, which is connected by a threaded insert 243 to a dose dial sleeve 244, where rotation of the dose dial sleeve 244 by the dosage knob 242 causes the selected dose to be displayed in a dosage window 212 in the housing 210 and causes a clicker 250 to interact with the drive sleeve 234 via a spring clutch 252. Together, the dosage knob 203, dose dial sleeve 230, and clicker 250 are a dose setting mechanism. The dose dial sleeve 244 is arranged around the clicker 250, which includes a feedback mechanism 251 that generates a tactile or audible feedback with rotation of the dose dial sleeve 244. The clicker 250 is coupled to the drive sleeve 234 with a metal clutch spring 252.

A last dose nut 260 (LDN) is provided on the drive sleeve 234. The last dose nut 260 advances with each dose dispensing operation to track the total medicament remaining in the cartridge 224. The trigger button 236 is depressed to activate a dose dispensing operation of the drug delivery device 200. The drive sleeve 234 includes flanges 262 and 264 that project from the drive sleeve. For instance, the flanges may be radial flanges. The LDN 260 is a threaded part, and suitably a half nut. The drive sleeve includes a threaded bolt section that typically extends between the two flanges. As the drive sleeve is rotated by corresponding rotation of the dose setting mechanism, the LDN 260 is caused to move along the drive sleeve by cooperation of the respective threads. The LDN is suitably arranged to move from flange 262, which is a minimum flange indicating the starting position of the LDN when the LDN abuts the flange and the cartridge is full. The LDN iteratively moves along the drive sleeve as each dose is injected. The LDN advances in response to rotation of the dose setting mechanism but does not translate relative to the drive sleeve as the drive sleeve is driven during the dose dispensing operation. The LDN abuts the other flange, which is a maximum flange that prevents the LDN from moving and consequently prevents the dose dialled mechanism from dialling in a dose that would exceed the dose remaining in the cartridge.

While the dose setting mechanism is illustrated as the dosage knob 242, dose dial sleeve 244, and the clicker 250, as described above, one skilled in the art will appreciate that any number of different dose setting mechanisms that are routine in the art for the purposes of setting a dose of a drug delivery device and aspects of the present disclosure are compatible with other such dose setting mechanisms. Similarly, while the dose dispensing mechanism is illustrated as including the piston rod 232, drive sleeve 234, trigger button 236, one skilled in the art will appreciate that a number of different dose dispensing mechanisms (e.g., drive mechanisms) are known in the art for the purposes of delivering or dispensing a dose of a drug delivery device and aspects of the present disclosure are compatible with other such dose dispensing mechanisms.

Continuing with the operation of the drug delivery device 200, turning the dosage knob 236 causes a mechanical click sound to provide acoustic feedback to a user by rotating the dose dial sleeve 244 with respect to the clicker 250. The numbers displayed in the dosage display 212 are printed on the dose dial sleeve 244 that is contained in the housing 210 and mechanically interacts with the drive sleeve 234 via the metal spring clutch 252. When the injection button 236 is pushed, the drug dose displayed in the display 212 will be ejected from the drug delivery device 200. During a dose setting operation, the drive sleeve 234 is helically rotated with the dose dial sleeve 234 spiralling outwardly in the distal-proximal direction. When the injection button 236 is pushed, the drive sleeve 234 is released and advanced distally, which causes rotation of the piston rod 232. The rotation of the piston rod 232 drives the pressure plate 237 against the stopper 228 of the cartridge 224, which drives the stopper 228 into the cartridge 224 to expel the medicament from the cartridge 224. A more detailed description of a representative drug delivery device is described in U.S. Pat. No. 7,935,088 B2, issued 3 May, 2011.

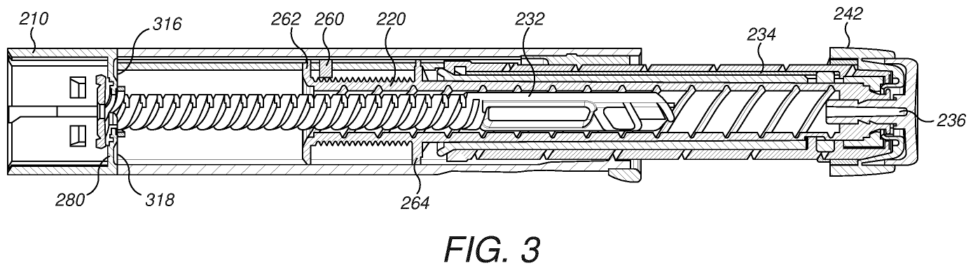

FIG. 3 shows the drug delivery device 200 at the end of a dose setting operation and prior to a dose dispensing operation, where the dose dial sleeve 244 and the drive sleeve 234 have been helically rotated with respect to the housing 210 and a threaded end 233 of the piston rod 232 to set the dose. The last dose nut 260 is shown advanced along the drive sleeve 234 from an initial position to a position indicative of the dose remaining in the drug delivery device 200. Upon dose dispensing of the injection button 236, the drive sleeve 234 advances into the housing 210 and a bearing nut 280 induces rotation of the piston rod 232. The bearing nut 280 sits fixed inside the housing 210 and has a threaded engagement with the piston rod 232. As the piston rod 232 rotates, the piston rod 232 is screwed forward (relative to the housing 210) because the bearing nut 280 cannot move. The rotation of the piston rod 232 drives the piston rod 232 and the pressure plate 237 proximally in the proximal-distal direction to drive the stopper 228 into the cartridge 224. Once dispensed, the drive sleeve is in a non-dose dialled position.

A medicament dose to be ejected from injection device 200 can be selected by turning the dosage knob 242, and the selected dose is then displayed via dosage window 212, for instance in multiples of International Units (IU). An example of a selected dose displayed in dosage window 12 may be ‘30’ IUs, as shown in FIG. 1. It should be noted that the selected dose may equally well be displayed differently, for instance by means of an electronic display.

Turning the dosage knob 242 causes a mechanical click sound to provide acoustic feedback to a user. The numbers displayed in dosage window 212 are printed on the sleeve 244 that is contained in housing 210. When needle 206 is stuck into a skin portion of a patient, and then injection button 236 is pushed, the medicament dose displayed in display window 212 is ejected from the injection device 200. When the needle 206 of the injection device 200 remains for a certain time in the skin portion after the injection button 236 is pushed, a high percentage of the dose is actually injected into the patient's body. Ejection of the medicament dose also causes a mechanical click sound, which is however different from the sounds produced when using dosage knob.

Whilst a pen injection device is briefly described, other injection devices are envisaged, as is known in the art.

In exemplary embodiments, a passive electronic arrangement 300 is included on the injection device. The passive electronic arrangement can be measured by a signal to indicate information that can correlate to a position of a part of the dose dispensing mechanism, or a displacement of a part of the dose setting mechanism, or a displacement of the stopper. A part of the passive electronic arrangement 300 is operatively connected to a moveable component, wherein said movement of said part causes a change in an electrical characteristic of the passive electronic arrangement. Thus, the passive electronic arrangement can be measured to provide a displacement of said part. Here, the displacement is calculated as a difference between a position of a respective part before an injection, compared to the position of said part after injection. Typically, the displacement is measured as a linear distance along the axis of the injection device.

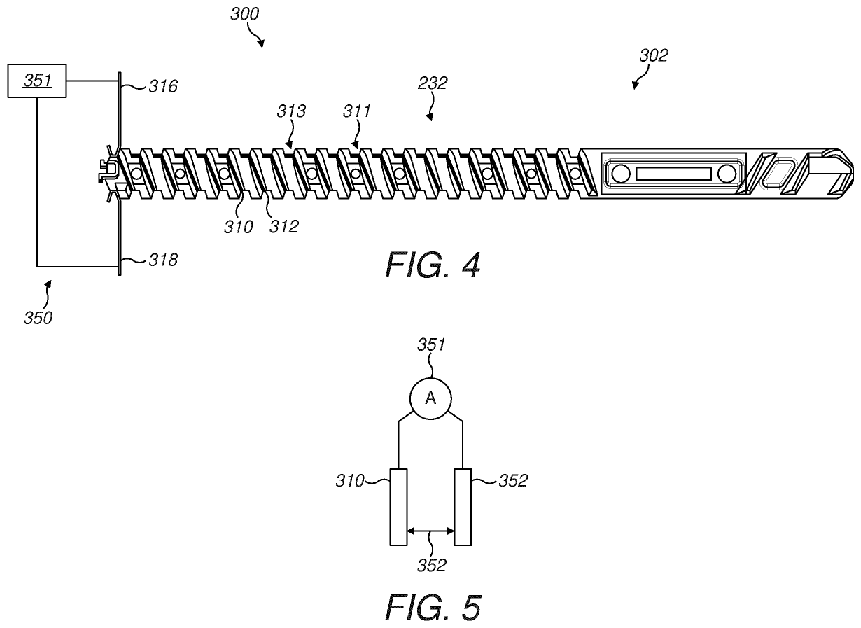

One example of a suitable passive electronic arrangement is a variable resistor 302 including a conductive electrode disposed on a track and wherein the conductive electrode is connected to a moveable component of the dose dispensing mechanism or the track is connected to the moveable component of the dose dispensing mechanism. Suitably, as shown in FIGS. 4 and 5, which are illustrations of a dose dispensing mechanism including conductive electrodes 310, 312 provided in individual tracks 311, 313 forming a variable resistor 302 for use in a dose tracking mechanism. As will be appreciated, the variable resistor 302 may be connected to a number of the moving parts of the injection device. The change in resistance corresponding to a change in length of the variable resistor is used as a measure of the displacement of the part.

One suitable aspect is based on using the change in length of the variable resistor 302 to indicate a displacement in the position of the piston rod 232 (e.g., a leadscrew), which is a key component of the dose dispensing mechanism of the drug delivery device 200 for use in expelling a dose of medicament. In dispensing a dose, the position of the piston rod 232 changes with respect to a bearing nut 280 by rotating with respect to the bearing nut 280, and thus moving proximally along the axis of rotation. By arranging two terminals of the variable resistor to connect via a variable length of resistive track, the length of resistive track between the terminals being varied by the position of the piston rod 232, a signal can be applied across the terminals and the control unit used to measure the resistance of the passive electrical arrangement. The signal in this case may be a direct current signal (i.e., a non-varying signal) of a particular voltage, but it may alternatively be an alternating current signal.

In more detail, FIG. 4 shows a piston rod 232 with embedded conductive elements 310, 312 and stationary wipers 316, 318 (e.g., conductive brushes), forming a variable resistor 302. The conductive elements form the resistive track and the stationary wipers act as the terminals of the variable resistor with the length of the resistive track connecting the terminals varying as the piston rod moves. Thus, the resistance of the passive electrical arrangement changes as the stationary wipers 316, 318 moves along the embedded conductive elements 310, 312. The piston rod 232 has a thread including two parallel oriented grooves 311, 313 helically extending along the axis of the piston rod 232. The conductive elements 310, 312 are embedded along the length of each of the two parallel oriented grooves 311, 313 without interfering with each other, except that they are electrically connected at one end of the grooves to create an open circuit across the brushes 316, 318. It will be appreciated that the conductive elements may alternatively be applied to peaks between the grooves.

In operation, the piston rod 232 is driven proximally by the drive sleeve 234, and the grooves 311, 313 are threaded through the bearing nut 280, such that the proximal movement of the drive sleeve 234 rotates the piston rod 232 causing it to pass through the bearing nut 280. The stationary wipers 316, 318 are disposed on the bearing nut 280 or otherwise fixed to the housing 210. A signal is applied to the variable resistor 302, and the resistance of the variable resistor is measured therefrom (e.g. by dividing the applied voltage by the current measured to flow through the variable resistor). As explained herein, suitably, the signal is a non-varying signal or an alternating/varying signal that is generated by a signal generator and associated control circuitry provided on an accessory and as part of a control unit.

The resistance across the wipers 316, 318 changes because of the change in total length of the conductive elements 310, 312 between the brushes 316, 318. For example, as shown in FIG. 3, the stationary brushes 316, 318 contact the conductive elements 310, 312 close to the proximal end of the grooves 311, 313. The conductive elements 310, 312 are electrically connected together at either the proximal end or distal end of the grooves 311, 313, but not both. If at the distal end, the electric path from one brush 316 to the other wiper 318 is down the entire length of the first groove 311 and back down the entire length of the second groove 313, the piston rod 232 is in a condition which represents the highest resistance configuration of the system. As the piston rod 232 is driven though the bearing nut 280, the wipers 316, 318 move along the grooves 311, 313, and the resistance across the wipers 316, 318 decreases as the total length of the conductive elements 310, 312 between the wipers 316, 318 decreases.

Alternatively, if the conductive elements 310, 312 are electrically connected together at the proximal end, then the opposite configuration is true, and the resistance across the wipers 316, 318 is at a minimum as shown, and increases as the piston rod 232 is driven though the bearing nut 280 and the total length of the conductive elements 310, 312 between the wipers 316, 318 increases.

In some instances, each specific resistance value represents one position of the piston rod 232 and therefore the resistance corresponds to an amount of the dose expelled from the cartridge 224 by the piston rod 232. In other instances, a change in the resistance corresponds to a change in position and is therefore proportional to the amount of medicament. Therefore, a relative change in resistance as compared to an initial resistance (e.g., before injection, or before a first use) corresponds to a measure for the medicament amount that has been expelled.

In some embodiments, the variable resistor 302 is used to modify the resonance frequency of an antenna of an RFID device 350 such that the change in resistance causes a corresponding change in the resonance frequency of the RFID device 350. Typically, the RFID device 350 includes an RFID chip 351 and an antenna 353. The passive electronic arrangement, for instance the variable resistor 302, forms a part of the antenna's circuit. In operation, the antenna 353 absorbs an incoming wireless reader signal from an external RFID reader (not shown) and forms a weak magnetic field, which creates a current in the antenna to provide power to the RFID chip 351. The RFID chip 351 includes a memory, which stores, for example, information related to the drug delivery device or a medicament contained therein. Upon power being provided to the RFID chip 351, the RFID generates a response signal in the antenna 353, which transmits the information from the RFID chip's 351 memory as a wireless signal. This wireless signal can be received by the external RFID reader that sent the reader signal, or by another device close by. Here, the RFID device 350 is used to determine the resistance of the variable resistor 302 by modulating the resonance frequency of an RFID device as a function of the position of the plunger rod. In FIG. 4, the RFID device is shown connected across the wipers.

FIG. 5 is a schematic of an alternative configuration, where the RFID device 350 is connected across the closed end of the conductive elements 310, 312, and a wiper 352 completes the circuit across the conductive elements 310, 312 at a variable location along the grooves 311, 313. In an example embodiment, an electrical property (e.g., resistance) of an RFID device's circuit is varied depending on the position of the last dose nut of the injection device. For example, the last dose nut includes a wiper and the thread on which it travels includes a galvanic/conductive track that has a certain resistance. The resistive value varies with the position of the last dose nut. Adding this resistance to the RFID circuitry would result in a slightly modified frequency. The value of the modified or detuned frequency can be determined by an RFID reader when receiving the signal. The amount of detune is proportional to the distance the last dose has travelled along the thread. As the frequency is varied with the position of the last dose nut, each position can be identified by a certain amount of detuned frequency. In some instances, the system can be calibrated during manufacturing, when the resistance of the track is known. In some instances, the frequency difference in relation to the initial frequency is taken as a measure and the difference is used to calculate an amount of medicament delivered or remaining.

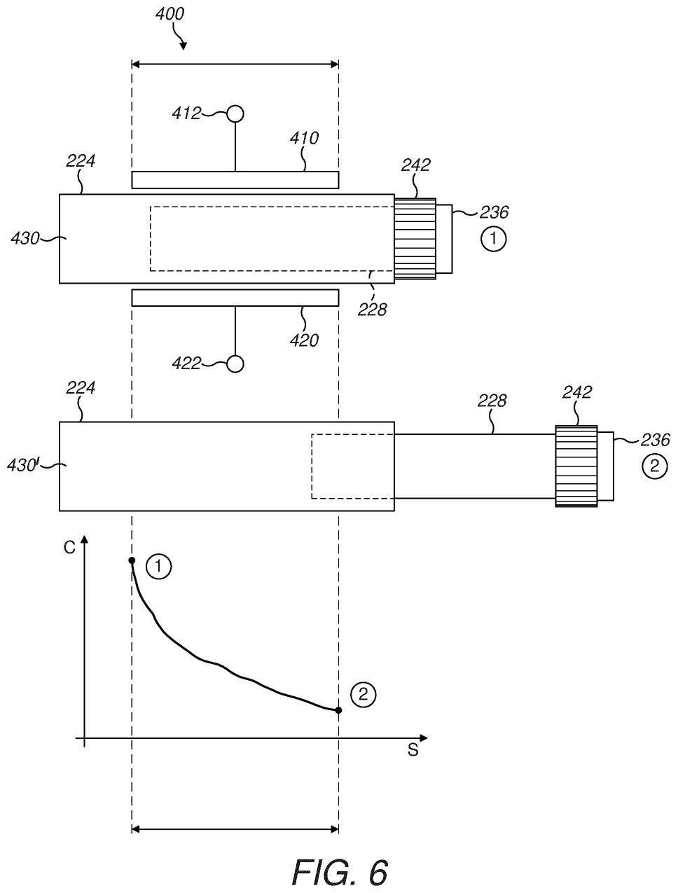

In a further suitable example of a passive electronic arrangement, FIG. 6 discloses an example wherein a capacitive sensor 400 is used to allow detection of displacement of the dose setting mechanism. Alternatively, by placing the capacitive sensor 400 to monitor the cartridge, the capacitive sensor can be used to detect the volume of medicament in the chamber of the cartridge.

With reference to FIG. 6, a simplified schematic illustration of the injection device and passive electrical arrangement for capacitive sensing of the status of the injection device is shown with reference to the cartridge 224 and a movable component 228.

Operatively connected to the movable component, as herein described, is the rotatory knob 242 for dosage selection and a dispense button 236. By rotating the knob 242, a user may select the dosage to be dispensed. The dispense button 236 can then be activated to move the movable component as a medicament is dispensed.

The outside of the cartridge 224 includes two metallic layers or plates 410, 420 arranged opposite to each other. The metallic layers or plates 410, 420 are connected in a circuit so as to form a capacitor, which is referred to in this specification as a capacitive sensor. Both layers 410, 420 may cover only a part of the outside of the cartridge 224, and each may include an electrical connector 412, 422 for connection with the control unit of the injection device. The metallic layers or plates 410, 420 may extend along the longitudinal axis of the housing 210 over a range, which includes nearly the entire displacement of the movable component 228 inside the housing 210, as shown in FIG. 6 by the double arrow at the top and the dashed vertical lines. A processor, as herein described, is suitably housed in an accessory along with a power source. Here, the processor causes an indication of the capacitance to be measured, e.g., by applying an alternating signal to the capacitive sensor and detecting a phase difference between the voltage and the current components of the signal, or by causing the application of a charging (direct current) signal to the capacitive sensor and then monitoring voltage decay resulting from capacitive discharge of the capacitive sensor through a resistive element within the accessory or the injection device.

The displacement of the stopper 228 with regard to the cartridge 224 by rotating the rotary knob 242 depending on the selected dosage and activating the dispense button 236 alters the medicament volume within the cartridge 224 from a larger fluid volume 430′ inside the cartridge 224 and shown at the bottom of FIG. 6 to a smaller fluid volume 430.

The alteration of the fluid volume 430 and the displacement of the movable component 228 inside the cartridge 224 influences the dielectric constant of the dielectric layer formed between the metallic layers 410, 420. This again results in a change of the capacitance of a capacitor formed by the metallic layers 410, 420 and the dielectric layer between them and formed by the fluid volume 430, the cartridge 224, and the movable component 228. The diagram at the bottom of FIG. 6 represents the change of the capacitance over the displacement of the movable component 228. At position (1), the capacitance is high since the movable component 228 is entirely moved into the cartridge 224 and the fluid volume 430 is as small as possible. Thus, the dielectric constant is higher than at position (2), where the movable component 228 is moved out of the cartridge 224 and the fluid volume 430′ is as large as possible. The capacitance therefore decreases from position (1) of the movable component 410 to position (2) of the movable component 410. This change of the capacitance is measurable and may be used for informing a user of a status of the injection device.

In exemplary embodiments, the metallic layers 410, 420 are provided in the label 211 affixed to the injection device. Here, the metallic layers 410, 420 are formed in separate parallel zones on the label 211 and when the label 211 is wrapped around the respective part of the injection device, the metallic layers 410, 420 form the opposed metallic layers 410, 420. For example, each metallic layer 410, 420 may form a half cylinder or portion of a cylinder. The label 211 may also include a shield to shield the metallic layers from external electromagnetic pulses.

The moveable part may include a metallic portion to increase the differentiation between dielectric constant as the part moves further into the space between the metallic layers. Typically, other parts such as the housing 210 and cartridge assembly 220 are made of plastics, for example ABS (Acrylonitrile butadiene styrene) or POM (Polyoxymethylene).

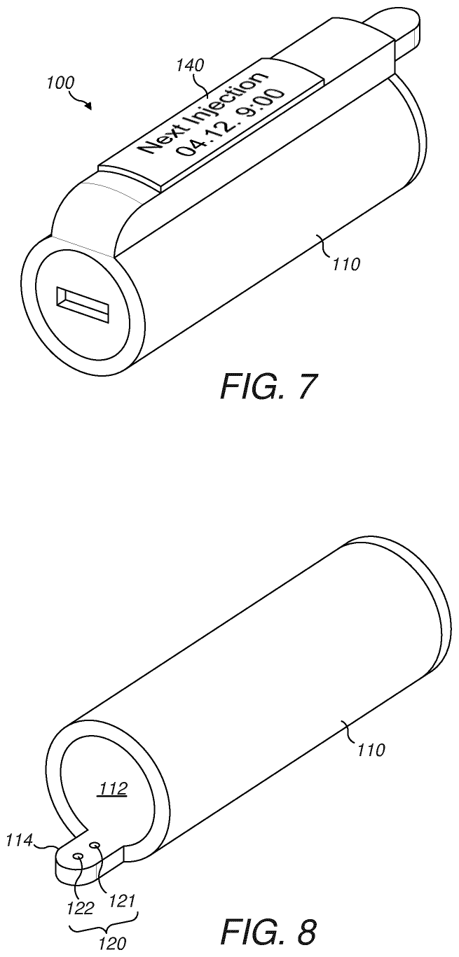

Referring to FIG. 7, an accessory 100 is shown. The accessory 100 is adapted to be attached to the injection device 200. Suitably, the accessory 100 replaces the cap 203 of the injection device. The accessory 100 includes a body 110 housing a control unit, a connector 120 and an optional display 140.

The body 110 forms a cavity 112 to receive a distal end of the injection device. The cavity has a closed distal end and an open proximal end. The injection device is inserted by relative movement of the injection device and the accessory 100 along the longitudinal axis. The body is sized so as to cover a distal end of the injection device. Suitably, the accessory 100 covers a substantial part of the distal end, for instance, the accessory covers the cartridge assembly 220. Here, the proximal opening of the accessory may be arranged to connect with the injection device, for instance the housing or cartridge holder. The accessory and injection device may include a press fit such that the accessory has a positive stop and cooperation between the respective parts provides a positive retaining force between the accessory and injection device when assembled together.

The accessory is arranged to be removable from a first injection device before an injection and replaced after the injection as is known with the cap 203 and to substantially replace the function of the cap 203. The accessory is also suitably arranged to be removeably attached to a second and subsequent injection device. Thus, the accessory can be reused. Here, because the accessory includes operational parts of the dose tracking mechanism, the operational parts can be reused between injection devices and in particular between disposable injection devices thereby saving resources.

The connector 120 is an electrical connector to electrically couple and decouple the accessory 100 to the injection device. As explained herein, the injection device includes a passive electronic arrangement. The connector 120 allows the control unit housed in the accessory 100 to connect to the passive electronic arrangement in the injection device. Thus, the connector 120 of the accessory 100 is coupled electrically to the control unit, for instance by conductive tracks or wiring within the body 110. Likewise, a corresponding connector is provided on the injection device, wherein the connector is electrically coupled to the passive electronic arrangement, for instance by conductive tracks or wiring through the label or housing.

In FIG. 8, the connector 120 is show as a first electrode 121 and a second electrode 122. This provides two electrical connection lines. It will be appreciated that based on the specific passive electronic arrangement adopted, further electrodes may be provided, or only one electrode may be provided as required. Suitably, the injection device also includes corresponding electrodes, wherein the electrodes form a part of the passive electronic arrangement.

Suitably each electrode 121, 122 is a conductive pad or the like as is known in the art. Here, the conductive pads on the accessory and corresponding conductive pads on the injection device are arranged to contact each other when the accessory is attached to the injection device. As shown for example in FIG. 9, the conductive pads therefore come in to contact by sliding over each other as the accessory is attached to the injection device.

It will be appreciated that the body 110 of the accessory houses the connector 120 so as to be aligned with the respective connector on the injection device. Where the respective connector on the injection device is arranged spaced from the part of the injection device covered by the accessory when attached, the accessory and as shown in FIG. 8 may include a projection 114 to extend the body 110 to cover said connector.

Referring back to FIG. 7, the accessory includes an optional display. The optional display 140 is housed in the body 110 of the accessory, for instance on an upper side (or user facing side). The display 140 may be an e-ink display module, which requires power to change the display but is capable of displaying images (such as text) for periods without power. The power and control for the display 140 are provided by the control unit of the accessory 100. The display can be controlled to display information of the injection or of the next injection and may include an area to display an indication of the last dose dialled/dispensed as part of the dose tracking mechanism.

As will be described in relation to FIG. 11, the control unit 150 of the accessory may include electronic modules such as a controller 151, power supply 190, communications module 170, and memory 160. The body may also provide one or more switches 130 that can be operated to control one or more of the processing functions.

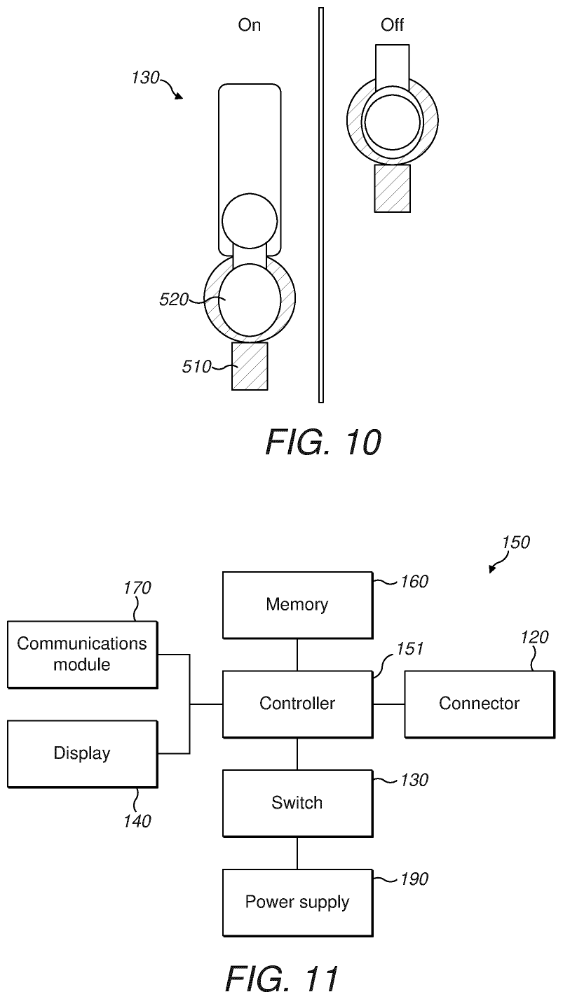

The switch 130 may be a manually-operable switch. Here, the switch 130 is manually activated to indicate one or more stages of an injection process. For instance, the switch may be activated after the accessory is reattached to the injection device following an injection. The activation of the switch thereby indicates an injection has occurred and the control unit is therefore arranged to be activated to interrogate the passive electronic arrangement in order to determine a post-injection location of the monitored part. Whist a pre-injection measurement may be activated, suitably the post injection location of the part from a previous injection is used as the first location so that the displacement of the part and therefore injected dose can be determined. Alternatively, and referring to FIG. 10, the switch may be automatically activated.

In FIG. 10 one of the respective accessory or injection device includes a catch 510 and the other a projection 520. As the accessory is attached to the injection device by movement along the longitudinal axis, the catch 510 catches or releases the projection 520. In FIG. 10, the catch is shown as a resilient claw such that the catch can push and pull the projection. As the catch is moved towards the projection, the catch abuts the projection. The projection may move to the other state before the accessory completes its movement such that further movement of the accessory to its attached position causes the claw to expand to capture the projection. The resilient force of the claw is such that when the accessory is removed from the injection device, the claw maintains a capture of the projection, moving the projection to the other state, before the claw releases the projection as further movement of the accessory removes the accessory form the injection device.

Movement of the projection activates the switch. Thus, the switch can be automatically activated by attaching and detaching the accessory from the injection device. Activation of the switch ‘wakes up’ the control unit thus preserving power. In FIG. 10, the switch 130 is shown as being closed (‘on’) when the accessory is detached and open (‘off’) when the accessory is reattached. It will be appreciated that the states can be alternated and/or the control unit can be arranged to move in to a ‘sleep’ state after a period of being on. In one embodiment, attaching the cap wakes the control unit up. Here, placing the cap on the injection device triggers the control unit to interrogate the passive electrical arrangement to determine a position of the moveable part being monitored. As well as triggering a measurement, replacing the accessory can trigger the control unit 150 to display the measurement or transmit the measurement to a remote device. The control unit can be arranged to power down straightway or after a short delay.

As shown in FIG. 11, the control unit 150 includes a controller 151 to control the dose tracking mechanism. Memory 160 may be provided as required. The controller controls the optional display 140 to display an image. Whilst the controller 151 may include a processing module to complete the dose tracking steps, preferably, the accessory includes a communications module 170 for communicating the acquired displacement information to a remote device for image processing. Here, the communications module is any suitable wireless communications module such as a Bluetooth, Wi-Fi, IRDA, NFC module or other short- or medium-range communications module. The switch 130 may be used to control the controller to establish a connection through the communications module 170 with the remote device. It will be appreciated that where the switch has alternative functions, the switch may be the same switch or there may be a plurality of switches each with their own function. Here, the switches may also be provided on the injection device where appropriate or necessary.

A power supply 190 is provided in the accessory to provide power to the respective parts. For instance the body 110 defines a battery compartment and the power supply is a battery inserted and electrically connected within the compartment.

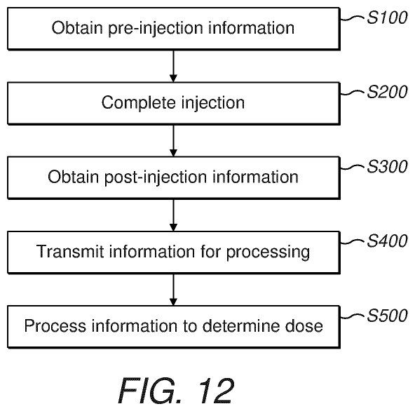

Referring to FIG. 12, a method of managing a dosage regime is shown. The method includes, at step S100, obtaining positional information of a part at a pre-injection position. Here, the control unit is activated to provide a signal to the passive electronic arrangement via the electrical connection between the accessory and injection device. The pre-injection information is a record indicative of a position of the respective part before an injection step is completed at step S200. The obtaining the pre-injection information may be triggered by operation of the switch 130, or alternatively, post-injection information of a previous injection may be used or recalled from memory.

Step S200 includes completing an injection step. The injection step includes dialling in a dose to be injected using the dose setting mechanism and activating the dose dispensing mechanism to dispense the medicament.

At step S300 post-injection information is obtained. Again, the post-injection information is obtained by the control unit providing a signal to the passive electronic arrangement via the electrical connection and may be triggered to obtain the information by operation of a switch.

Suitably, the switch is triggered automatically by replacement of the accessory on the injection device which generally indicates an injection process in complete.

At step S400 an optional communications step is completed to transmit the information for processing on a remote device. Here, the post-injection information and optionally the pre-injection information are transmitted to the remote device for image processing by a processor. Alternatively, the processor is included as part of the control unit housed on the accessory.

At step S500 the information is processed to determine a displacement of the part and therefore a determination of the medicament dispensed. Here, the processor processes the information and calculates the dose dialled measurement or dose dispensed measurement in the processing step S500. Here the dose dialled measurement may be a calculation including the displacement of a part of the injection device and other parameters of the dose dispensing mechanism such as screw pitch and diameter. After the processing step calculates the dose dialled measurement, the processor may transmit the calculated measurement or other information of the injection regime to be displayed by the display 140.

Alternatively, step S400 may be omitted and S500 may be performed by the accessory. In these embodiments, the calculated dose may or may not be transmitted to another device.

The user may use the calculated measurement displayed on the display 140 in a manual log book. However, in exemplary embodiments, the remote device includes a software application program to electronically log the calculated dose dialled measurement and optionally other injection parameters. For instance, the dosage management is implemented through a computer or the like. For instance, as an application on a smartphone, or tablet, or the like, wherein the application monitors and alerts the user to the time and dosage of the injections. The application can also be used to input and record the injection parameters, for instance, to automatically log the time and date of the injection.

According to the above, the accessory 100 can be attached to an injection device 200 and in doing so, the accessory can be controlled to interrogate the passive electronic arrangement in the injection device. Since each injection device need only require the passive electronic arrangement as the control unit is housed in the accessory and can be reused between devices, the resources are reduced. The pre-injection and post-injection information are processed to calculate a dose dispensed measurement, which is used in the dose management regime. By electronically logging or displaying the measurement for electronic logging, the dose management regime can be improved, for instance, by improving the recording accuracy or automation of the dose dialled measurement.

Although claims have been formulated in this application to particular combinations of features, it should be understood that the scope of the disclosure also includes any novel features or any novel combinations of features disclosed herein either explicitly or implicitly or any generalisation thereof, whether or not it relates to the same conceptual approach as presently claimed in any claim and whether or not it mitigates any or all of the same technical problems as does the present disclosure. The applicant hereby gives notice that new claims may be formulated to such features and/or combinations of features during the prosecution of the present application or of any further application derived therefrom.

Although several embodiments have been shown and described, it would be appreciated by those skilled in the art that changes may be made in these embodiments without departing from the principles of the disclosed solution concepts, the scope of which is defined in the claims.

The injection device may include a cartridge containing a liquid drug or medicament. In instances, by pressing the injection button a portion thereof may be expelled from the cartridge according to the dialled or pre-set amount.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-15. (canceled)

16. An apparatus comprising:
an accessory and an injection device wherein:
the accessory attaches and detaches to the injection device, the accessory housing a control unit;
the injection device comprises a passive electronic arrangement; and
the control unit is configured to use the passive electronic arrangement to determine information correlating to a dose of medicament injected.

17. The apparatus of claim 16, wherein the accessory forms a cap that attaches to and covers an end of the injection device.

18. The apparatus of claim 16, wherein the injection device comprises a cartridge containing the dose of medicament.

19. The apparatus of claim 16, wherein one of the accessory and injection device comprises a catch and the other of the accessory and injection device comprises a part arranged to be caught by the catch,
wherein one of the catch or the part moves relative to the other of the catch or the part to activate a switch of the control unit and the switch being arranged to, in response to attachment or detachment of the accessory from the injection device, trigger the control unit to use the passive electronic arrangement to determine the information correlating to the dose of medicament injected.

20. The apparatus of claim 16, wherein the accessory houses a first connector and the injection device comprises a second connector for cooperating with the first connector of the accessory to electrically connect the control unit to the passive electronic arrangement.

21. The apparatus of claim 20, wherein the control unit of the accessory comprises a controller and a power supply, and
the controller controls the power supply to apply a signal to the passive electronic arrangement via the connector.

22. The apparatus of claim 20, wherein the control unit of the accessory comprises a controller and a communications module, and
the controller controls the communications module to communicate with a remote device.

23. The apparatus of claim 20, wherein the control unit of the accessory comprises a controller and a display, and
the controller controls the display to display the information correlating to the dose of medicament injected.

24. The apparatus of claim 16, wherein the passive electronic arrangement comprises a variable electronic resistor or wherein the passive electronic arrangement comprises a capacitive sensor.

25. The apparatus of claim 24, wherein the passive electronic arrangement is the capacitive sensor and the capacitive sensor comprises first and second opposed metallic layers, and
wherein the metallic layers are formed in a label applied around a cartridge assembly or housing of the injection device.

26. An accessory comprising:
a body housing a control unit,
wherein the accessory attaches to an injection device including a passive electrical arrangement, and
the control unit uses the passive electrical arrangement to determine information correlating to a dose of medicament injected.

27. The accessory of claim 26, wherein the control unit includes a switch, and
the accessory includes one of a catch or a corresponding part to be caught by the catch and movement of one of the catch or the corresponding part activates the switch.

28. The accessory of claim 26, wherein the control unit comprises a controller and a communications module, and
the controller controls the communications module to communicate with a remote device.

29. The accessory of claim 26, wherein the control unit comprises a controller and a display, and
the controller controls the display to display the information correlating to the dose of medicament injected.

30. The accessory of claim 26, wherein the control unit comprises a controller and a power supply, and
the controller controls the power supply to apply a signal to the passive electronic arrangement.

31. The accessory of claim 26, wherein the accessory forms a cap that attaches to and covers an end of the injection device.

32. An injection device comprising:
a passive electronic arrangement that can be used by the accessory of claim 26, to provide information corresponding to a position of a part that moves during an injection.

33. The injection device of claim 32, wherein the passive electronic arrangement comprises a variable electronic resistor or wherein the passive electronic arrangement comprises a capacitive sensor.

34. The injection device of claim 33, wherein the passive electronic arrangement is the capacitive sensor, and
the capacitive sensor comprises first and second opposed metallic layers and wherein the first and second opposed metallic layers are formed in a label applied around a cartridge assembly or housing of the injection device.

35. The injection device of claim 32, further comprising a cartridge containing medicament.
